(19) United States
(12) Patent Application Publication
(10) Pub. No.: US 2015/0346188 A1
(43) Pub. Date: Dec. 3, 2015

(54) STABILIZED NANOPORE AND MICROPORE STRUCTURES AND METHODS FOR MAKING AND USING THE SAME

(71) Applicant: IBIS BIOSCIENCES, INC., Carlsbad, CA (US)

(72) Inventor: Mark W. Eshoo, San Diego, CA (US)

(21) Appl. No.: 14/653,963

(22) PCT Filed: Dec. 20, 2013
(86) PCT No.: PCT/US13/77147

§ 371 Date: Jun. 19, 2015

Related U.S. Application Data
(60) Provisional application No. 61/740,966, filed on Dec. 21, 2012.

(51) Int. Cl. G01N 33/487

(52) U.S. Cl. CPC G01N 33/48721

(57) ABSTRACT

The invention relates generally to nanopore and micropore structures, methods for making the structures and use of the structures. In particular, the invention provides nanopore structures (e.g., containing lipid bilayers) in combination with one or more osmoprotective compounds and methods of making and using the same. Compositions and methods of the invention fmd use in a wide range of applications including molecular biology and medical science.

### CROSS-REFERENCE TO RELATED APPLICATIONS

The present Application claims priority to U.S. Provisional Application Serial No. 61/740,966 filed 21 Dec. 2012, the entirety of which is incorporated by reference herein.

### FIELD OF THE INVENTION

The invention relates generally to nanopore and micropore structures, methods for making the structures and use of the structures. In particular, the invention provides nanopore structures (e.g., containing lipid bilayers) in combination with one or more osmoprotective compounds and methods of making and using the same. Compositions and methods of the invention find use in a wide range of applications including molecular biology and medical science.

### BACKGROUND OF THE INVENTION

Characterizing polymers (e.g., polypeptides, proteins, protein fragments, nucleic acids or other polymer) exists as a crucial component of the life sciences (e.g., research, development, clinical diagnosis, etc.). For example, determining the nucleotide sequence of DNA and RNA (e.g., in a rapid manner) remains a goal of researchers in biotechnology. Rapidly determining the sequence of a nucleic acid molecule is important for identifying genetic mutations and polymorphisms in individuals and populations of individuals, as well as for identifying the presence of microorganisms in a sample (e.g., via detection of microorganism specific nucleic acid).

### SUMMARY OF THE INVENTION

The invention relates generally to nanopore and micropore structures, methods for making the structures and use of the structures. In particular, the invention provides nanopore structures (e.g., containing lipid bilayers) in combination with one or more osmoprotective compounds and methods of making and using the same. Compositions and methods of the invention find use in a wide range of applications including molecular biology and medical science.

Accordingly, in one embodiment, the invention provides a device comprising one or more nanopores associated with one or more osmoprotectants. In some embodiments, the nanopores are in physical contact with one or more osmoprotectants. In some embodiments, the nanopores are utilized for sensing or characterizing molecules. The invention is not limited by the type of molecule characterized. Indeed, a variety of molecules may be characterized including, but not limited to, nucleic acids, proteins, protein fragments, amino acids, and the like. In a preferred embodiment, the molecules are nucleic acid. In a further preferred embodiment, the one or more osmoprotectants allows the device to be utilized in a manner not possible in the absence of the osmoprotectant. For example, in some embodiments, use of one or more osmoprotectants allows the device to be utilized at a more optimal (e.g., higher) salt concentration. The invention is not limited by the type of osmoprotectant utilized. Indeed, a variety of osmoprotectants may be used including, but not limited to, glycinebetaine, trehalose, sorbitol, sucrose, and 2-o-a-o-glucopyranosyl glycerol. In a preferred embodiment, the osmoprotectant is betaine. The invention is not limited by the type of betaine used. In some embodiments, the betaine is trimethylglycine. In some embodiments, the betaine is a phosphonium betaine. In some embodiments, the diameter of the nanopore is between 100 nm and 1 nm, although wider and narrower nanopores may be used. In some embodiments, a time-dependent transport property of the nanopore is superior in the presence of the one or more osmoprotectants compared to the property in the absence of the one or more osmoprotectants. In some embodiments, the time-dependent transport property is current, conductance, resistance, capacitance, charge, concentration, optical property, or chemical structure. In some embodiments, the one or more osmoprotectants allows a device comprising a nanopore or plurality of nanopores to operate at a higher efficiency than when in the absence of an osmoprotectant. In some embodiments, the device comprises a nanopore complex. In some embodiments, the complex comprises a substrate with a nanopore formed therein. In some embodiments, the complex further comprises a nucleic acid within the nanopore. In some embodiments, the complex comprises a lipid bilayer. In some embodiments, the complex comprises an enzyme. The invention is not limited by the type of substrate material. Indeed, a variety of materials may be used including, but not limited to, silicon, mica, polyimide or lipid. In some embodiments, the device is a nucleic acid (e.g., DNA or RNA) sequencing device.

The invention also provide the use of any one of the devices disclosed herein. For example, in some embodiments, a device disclosed herein is utilized to sequence nucleic acid (e.g., DNA or RNA).

### DETAILED DESCRIPTION OF THE INVENTION

The invention relates generally to nanopore and micropore structures, methods for making the structures and use of the structures. In particular, the invention provides nanopore structures (e.g., containing lipid bilayers) in combination with one or more osmoprotective compounds and methods of making and using the same. Accordingly, in some embodiments, the invention provides nanopore and/or micropore structures (e.g., containing lipid bilayers and/or that are useful for sensing and/or characterizing macromolecules (e.g., peptides, proteins, protein fragments, cells, DNA or RNA) as well as sequencing DNA or RNA) in combination with one or more osmoprotectants and methods of making and using the same. Compositions and methods of the invention find use in a wide range of applications including molecular biology and medical science. Devices and systems comprising one or more nanopores (e.g., electrically-addressable-solid-state nanopores) in combination with and/or in the presence of (e.g., in physical contact with) one or more osmoprotectants useful for sensing and/or characterizing macromolecules (e.g., amino acids, proteins, protein fragments, nucleic acid, etc.) as well as sequencing DNA or RNA are described.

Accordingly, in some embodiments, the invention provides a nanopore, a plurality of nanopores, and/or a device or system comprising a nanopore or a plurality of noanopores. A device or system comprising a nanopore may be made of any material known in the art including, but not limited to, silicon nitride, silicon oxide, mica, polyimide, or lipids. The device or system may comprise detection electrodes and/or detection integrated circuitry. The device or system comprising a nanopore or plurality of nanopores may simply be composed of material used to make the device/system, or it may further comprise one or more other materials (e.g., peptides, proteins, protein sequences, nucleic acids (e.g., DNA or RNA)) that can assemble or form a channel or structure within the device/system. In some embodiments, a nanopore within the device/system is constructed so as to have a dimension such that only a single stranded polynucleotide can pass through a nanopore at a given time, so that a double or single stranded polynucleotide can pass through a nanopore, so that neither a single nor a double stranded polynucleotide can pass through a nanopore, or so that more than one double stranded polynucleotide can pass through a nanopore. In some embodiments, a nanopore within a device/system is constructed so as to have a dimension such that only a single polymer (e.g., amino acid sequence (e.g., peptide, protein, or protein fragment) can pass through a nanopore at a given time. In other embodiments, a nanopore within the device/system is constructed so as to have a dimension such that multiple polymers (e.g., peptide, protein, protein fragments, nucleic acid) can pass through a nanopore.

The system/device comprising a nanopore or plurality of nanopores may further comprise other agents/molecules (e.g., proteins, enzymes, small molecules (e.g., that assist in the transport of a substance (e.g., peptide, protein, protein fragments, cells nucleic acid, etc.) through a nanopore)). For example, in some embodiments, a system/device comprising a nanopore or plurality of nanopores further comprises an agent that is capable of moving a molecule (e.g., a protein, polypeptide, nucleic acid, etc.) into, through or out of a nanopore. For example, in some embodiments, a system/device comprising a nanopore or plurality of nanopores further comprises an agent that moves a polynucleotide into, through or out of a nanopore. Exemplary agents include, but are not limited to, polymerases (e.g., DNA polymerase and RNA polymerase), helicases, ribosomes, and exonucleases. However, the invention is not so limited. For example, any agent that possesses one or more of the following traits may be used:

addition or removal of one nucleotide per turnover; no backtracking along a target polynucleotide; no slippage of the agent on the target polynucleotide (e.g., due to forces (e.g., from an electric field) employed to drive a polynucleotide to the agent); retention of catalytic function when disposed adjacent a nanopore; high processivity (e.g., the ability to remain bound to target polynucleotide and perform many (e.g., at least 100, at least 500, at least 1,000 or more rounds of catalysis before dissociating)).

An agent used within a device/system of the invention or in accordance with methods described herein can depend, in part, on the type of target molecule (e.g., DNA, RNA, polypeptide, etc.) being analyzed. For example, an agent such as a DNA polymerase or a helicase is useful when the target polynucleotide is DNA, and an agent such as RNA polymerase is useful when the target polynucleotide is RNA. In addition, the agent used will depend, in part, on whether the target polynucleotide is single-stranded or double-stranded. Those of ordinary skill in the art are able to identify appropriate agents useful according to the particular application.

Exemplary DNA polymerases include E. coli DNA polymerase I, E. coli DNA polymerase I Large Fragment (Klenow fragment), phage T7 DNA polymerase, Phi-29 DNA polymerase, thermophilic polymerases (e.g., Thermus aquaticus (Taq) DNA polymerase, Thermus flavus (Tfl) DNA polymerase, Thermus Thermophilus (Tth) DNA polymerase, Thermococcus litoralis (Tli) DNA polymerase, Pyrococcus furiosus (Pfu) DNA polymerase, VENT DNA polymerase, or Bacillus stearothermophilus (Bst) DNA polymerase), and a reverse transcriptase (e.g., AMV reverse transcriptase, MMLV reverse transcriptase, or HIV-1 reverse transcriptase). Other suitable DNA polymerases are known in the art.

Exemplary RNA polymerases include T7 RNA polymerase, T3 RNA polymerase, SP6 RNA polymerase, and E. coli RNA polymerases.

Exonucleases include, but are not limited to, Exonuclease Lambda, T7 Exonuclease, Exo III, RecJ₁ Exonuclease, Exo I, and Exo T).

Helicases include both RNA and DNA helicases. Helicases have previously been described in U.S. Pat. No. 5,888,792. Exemplary helicases include hexameric helicases such as the E-coli bacteriophage T7 gp4 and T4 gp41 gene proteins, and the E. coli proteins DnaB, RuvB, and rho (See, e.g., West S C, Cell, 86, 177-180 (1996)). Hexameric helicases unwind double stranded DNA in a 5′-3′ direction, which ensures a directional analysis of the DNA target molecules. In addition, some of the helicases have processive translocation rates in excess of 1000 nucleotides per second (See, e.g., Roman et al. J. Biol. Chem. 267:4207 (1992)). In addition, these hexameric helicases form a ring structure having an inside hole dimension ranging in size from 2-4 nanometers and an outside ring dimension of about 14 nanometers. The hexameric ring structure is formed and stabilized in the presence of Mg⁺² and some type of nucleotide (NMP, NDP or NTP).

Such agents (e.g., polymerases (e.g., DNA polymerase and RNA polymerase), helicases, ribosomes, and/or exonucleases) may be located on the cis or trans side of a device/system comprising a nanopore or plurality of nanopores. By “cis” is meant the side of a nanopore through which a polymer enters the pore or across the face of which the polymer moves. By “trans” is meant the side of a nanopore aperture through which a polymer (or fragments thereof) exits the pore or across the face of which the polymer does not move. By “polynucleotide” is meant DNA or RNA, including any naturally occurring, synthetic, or modified nucleotide. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, 2-thiocytidine as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP. In some embodiments, the agent is substantially immobilized, but need not be immobilized, adjacent the nanopore and/or inline with the nanopore by a matrix material, by chemically bonding with the structure using chemical bonding materials, or by any other appropriate means (e.g., noncovalent interactions). In some embodiments, the agent is wholly or partially disposed within a nanopore.

Matrix materials utilized to make a nanopore or plurality of nanopores of a device/system of the invention include, but are not limited to, natural polymers (e.g., agar, agarose, and other polysaccharide-based materials), synthetic polymers, and sol-gels. Synthetic polymers include, but are not limited to, polyacrylamides (e.g., polymerized chemically or through irradiation with UV light, X-rays or gamma rays). These types of matrices have been used to immobilize and entrap molecules for a variety of applications. For example, penicillin acylase has been shown to maintain enzymatic activity when embedded within acrylamide polymers having varying degrees of porosity and cross-linking (See, e.g., Prabhune A., and Sivaraman H., Applied Biochem. and Biotech. 30, 265-272 (1991), Wuyun G W, et al. Acta Chirnica Sinica, 60 504-508 (2002)). Alkaline phosphatase has been immobilized by physical entrapment with colloidal particles having functionalized surfaces comprising copolymers of acrylamide (See, e.g., Daubresse et al., Colloid and Polymer Science, 274, 482-489 (1996) and Daubresse et al., J. of Colloid and Interface Science, 168, 222-229 (1994)).

In some embodiments, agents are covalently linked to the matrix material in order to prevent the agents from diffusing away from a nanopore. This may be important when using lower density polymer matrices that enable larger substrate molecules (e.g., >1 kb polynucleotides) to freely migrate through the matrix material. The covalent linkage between the agent and matrix material may be formed by any one of a number of methods known in the art. Direct linkage between natural amino acid residues such as lysine or cysteine within the agent and the matrix material can be formed using chemical methods. The agents may be engineered to contain desired residues for specific linking chemistries. A synthetic linker having a defined reactive moiety (e.g., N,N′-methylenebisacrylamide) can be attached to the agent prior to immobilization so that the agent becomes linked to the matrix material during the matrix material formation process.

In some embodiments, a chemical bonding material is disposed on the structure to chemically bond (e.g., covalent or non-covalent bonding) the agent to the structure. The chemical bonding material is positioned so that the bound agent is adjacent and substantially inline with the nanopore. The bonding between the chemical bonding material and the agent can include, but is not limited to, bonding between amine, carboxylate, aldehyde and sulfhydryl functional groups on the agent and the chemical bonding material through linker or chemical conjugations involving reactive groups such isothiocyanates, acyl azides, NHS esters, sulfonyl chlorides, epoxides, carbonates, carbodiimides and anhydrides (See, e.g., G. T. Hermanson, Bioconjugate Techniques (1996), Academic Press, Inc., San Diego Calif.). Thus, the chemical bonding material can include, but is not limited to, compositions having groups such as isothiocyanates, acyl azides, NHS esters, sulfonyl chlorides, epoxides, carbonates, carbodiimides, and anhydrides.

The system/device comprising a nanopore or plurality of nanopores may further comprise one or more osmoprotectants. While an understanding of a mechanism of action is not needed to practice the invention, and the invention is not limited to any particular mechanism of action, in some embodiments, the one or more osmoprotectants stabilize components (e.g., agents recited above, peptides, proteins, enzymes, protein fragments, nucleic acids, etc.) of the device/system. For example, in a preferred embodiment, the presence of one or more osmoprotectants within a device/system comprising one or a plurality of nanopores of the invention allows the device/system to be utilized in a manner not possible in the absence of the osmoprotectant. For example, in some embodiments, the presence of one or more osmoprotectants stabilizes components of a nanopore device/system (e.g., lipid bilayers, enzymes, proteins, nucleic acids, etc.). For example, the presence of one or more osmoprotectants allows the use of a device/system comprising a nanopore at a higher/more optimal level (e.g., a higher salt concentration (e.g., that permits optimal enzyme activity)) than is possible in the absence of the osmoprotectant.

The presence of one or more osmoprotectants can also be used to stabilize lipid bilayer structures (e.g., thereby increasing the shelf-life of a device/system comprising a nanopore (e.g., a nanopore/lipid bilayer/enzyme complex)). For example, the use of one or more osmoprotectants in a device/system of the invention can increase the shelf-life by days, weeks, months or even years.

In some embodiments, the presence of one or more osmoprotectants allows a device/system comprising a nanopore to be usable at currents and/or voltages unattainable in the absence of the osmoprotectant. Similarly, in some embodiments, the presence of one or more osmoprotectants allows a device/system comprising a nanopore to be used with a medium (e.g., fluid medium) that is otherwise not usable or that is not optimal for use. For example, the medium located on either side of a nanopore (e.g., disposed in pools on either the cis or trans side of a nanopore) may be any fluid that permits mobility of molecules (e.g., peptides, nucleic acids, etc.) through the nanopore (e.g., polynucleotide mobility through a nanopore). In some embodiments, the medium is a liquid, aqueous solution or other liquid or solutions in which molecules (e.g., polypeptides, proteins, polynucleotides) can be distributed. When an electrically conductive medium is used, it can be any medium which is able to carry electrical current. Such solutions generally contain ions as the current-conducting agents (e.g., sodium, potassium, chloride, calcium, magnesium, cesium, barium, sulfate, and/or phosphate). Conductance across the nanopore can be determined by measuring the flow of current across the nanopore (e.g., from one end to the other) via the conducting medium. A voltage difference can be imposed across the barrier between the pools using electronic equipment. Alternatively, an electrochemical gradient may be established by a difference in the ionic composition of the two pools of medium on each side of the nanopore, either with different ions in each pool, or different concentrations of at least one of the ions in the solutions or media of the pools. Conductance changes can be detected and utilized to collect information about nanopore (e.g., transfer of molecules through the nanopore).

Detection of molecules that have traveled within a nanopore of a device/system of the invention is also provided. For example, time-dependent transport properties of the nanopore may be measured by any suitable technique. The transport properties may be a function of the medium used to transport molecules (e.g., polypeptides, polynucleotides, etc.), solutes (e.g., ions) in the liquid, the presence of one or more osmoprotectants, the chemical structure of a molecule (e.g., polypeptide, polynucleotide, etc.) traveling within the nanopore, and/or labels on the molecules. Exemplary transport properties include current, conductance, resistance, capacitance, charge, concentration, optical properties (e.g., fluorescence and Raman scattering), and chemical structure. In some embodiments, the presence of one or more osmoprotectants stabilizes and/or optimizes one or more of these transport properties (e.g., thereby enhancing and/or optimizing activity of a device/system comprising a nanopore or plurality of nanopores of the invention).

In some embodiments, the transport property is current. Suitable methods for detecting current in nanopore systems are known in the art, for example, as described in U.S. Pat. Nos. 6,746,594, 6,673,615, 6,627,067, 6,464,842, 6,362,002, 6,267,872, 6,015,714, and 5,795,782 and U.S. Publication Nos. 2004/0121525, 2003/0104428, and 2003/0104428. In another embodiment, the transport property is electron flow across the diameter of the nanopore, which may be monitored by electrodes disposed adjacent to or abutting on the nanopore circumference.

In one example of the use of a device/system comprising a nanopore or plurality of nanopores of the invention, characterization of polynucleotides using the device/system involves the use of two separate pools of a medium and an interface between the pools. The interface between the pools is capable of interacting sequentially with the individual monomer residues of a polynucleotide present in one of the pools. Measurements of transport properties are continued over time, as individual monomer residues of the polynucleotide interact sequentially with the interface, yielding data suitable to determine a monomer-dependent characteristic of the polynucleotide. The monomer-dependent characterization achieved by nanopore sequencing may include identifying characteristics such as, but not limited to, the number and composition of monomers that make up each individual polynucleotide (e.g., in sequential order). In one embodiment, use of one or more osmoprotectants described herein allows a device/system comprising a nanopore or plurality of nanopores of the invention to operate at a higher efficiency than when in the absence of an osmoprotectant.

A polynucleotide being characterized may remain in its original pool or a reaction product including it or fragments thereof may cross the nanopore into the other pool. In either situation, the target polynucleotide moves in relation to the nanopore, and individual nucleotides interact sequentially with the nanopore giving rise to a change in the measured transport properties (e.g., conductance) of the nanopore. When the polynucleotide does not cross into the trans side of the device, it is held adjacent the nanopore such that its nucleotides interact with the nanopore passage and bring about changes in transport properties, which are indicative of polynucleotide characteristics.

For example, in some embodiments, without use of an osmoprotectant, single stranded DNA molecules are driven through a nanopore by a voltage bias at a certain rate (e.g., about 1-2 nucleotides per microsecond, and dsDNA translocates through nanopores approximately two orders of magnitude faster). However, when an osmoprotectant is utilized, the intrinsic rate of transfer of single stranded DNA and/or double stranded DNA is modified (e.g., is slowed down to be resolvable, and/or increased in speed while retaining the ability to be resolved). Accordingly, the invention provides economically affordable systems/devices for nucleotide sequencing.

The rate of movement of a molecule (e.g., polynucleotide) with respect to a nanopore may be between 0 and 2000 Hz, desirably between 50-1500 Hz, 100-1500 Hz, or 350-1500 Hz, e.g., at least 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, or 1900 Hz and/or at most 1750, 1500, 1450, 1400, 1350, 1300, 1250, 1200, 1150, 1100, 1050, 1000, 950, 900, 850, 800, 750, 700, 650, 600, 550, 500, 450, 400, 350, 300, 250, 200, or 150 Hz. In some preferred embodiments, the rate is controlled by the use of one or more osmoprotectants and/or agents that move molecules (e.g., polynucleotides) at a substantially constant rate (e.g., during such time that the molecules are being characterized). In some embodiments, the range of rate of movement depends upon the presence of the osmoprotectant (e.g., the ability of the osmoprotectant to stabilize lipid bilayers (e.g., via enhanced salt tolerance)).

The intrinsic rate of movement of molecules caused by a particular osmoprotectant can be modified by changing the amount and/or type(s) of osmoprotectant utilized, as well as by other factors (e.g., changes in temperature, pH, ionic strength, the presence of cofactors, substrates, inhibitors, agonists, or antagonists, by the nature of the medium (e.g., the presence of nonaqueous solvents or the viscosity), by external fields (e.g., electric or magnetic fields), and hydrodynamic pressure). Such modifications may be used to start, stop, increase, decrease, or stabilize the rate of movement, which may occur during a particular characterization. In addition, such modifications may be used as switches, brakes, or accelerators, e.g., to start, stop, increase, or decrease movement of a molecule (e.g., polynucleotide). In alternative embodiments, external forces (e.g., electric or magnetic fields or hydrodynamic pressure) may be used to control the rate of movement. The rate of movement may be substantially slowed or even stopped (e.g., paused) before, during, or after analysis of a particular polynucleotide.

In some embodiments, the presence of one or more osmoprotectants allows a device/system comprising a nanopore to contain usable nanopores that are either smaller or larger than nanopores that are functional in the absence of the osmoprotectant. In some embodiments, the presence of one or more osmoprotectants allows the translocation of molecules (e.g., proteins, nucleic acids, cells, peptides, etc.) through a nanopore that would otherwise not occur (e.g., in the absence of an osmoprotectant). In some embodiments, the presence of one or more osmoprotectants allows the rate of translocation of molecules (e.g., proteins, nucleic acids, cells, peptides, etc.) through a nanopore that would otherwise not be achievable (e.g., in the absence of an osmoprotectant). For example, in some embodiments, the presence of one or more osmoprotectants allows for the movement of molecules (e.g., proteins, nucleic acids, cells, peptides, etc.) through a nanopore at a faster rate than is possible in the absence of the osmoprotectant. In some embodiments, the presence of one or more osmoprotectants allows for the movement of molecules (e.g., proteins, nucleic acids, cells, peptides, etc.) through a nanopore at a slower rate than is possible in the absence of the osmoprotectant.

The invention is not limited to any particular osmoprotectant. Indeed, a variety of osmoprotectants may be utilized including, but not limited to, glycinebetaine, betaine, trehalose, sorbitol, sucrose, and 2-o-a-o-glucopyranosyl glycerol. In a preferred embodiment, the osmoprotectant is a betaine. As used herein, a betaine referes to a neutral chemical compound with a positively charged cationic functional group (e.g., a quaternary ammonium or phosphonium cation (e.g., generally referred to as onium ions)) that bear no hydrogen atom and with a negatively charged functional group (e.g., a carboxylate group (e.g., that need not be adjacent to the cationic site)). Thus, in some embodiments, a betaine is a zwitterion. In some embodiments, the betaine is trimethylglycine. In some embodiments, the betaine is a phosphonium betaine.

The invention is not limited by the diameter size of a nanopore described herein. For example, any nanopore of the appropriate size may be used in a device/system and/or methods of the invention. The size of a nanopore or micropore is any size that is useful for a particular application. For example, in some embodiments, a nanopore comprises a diameter in the range of 100 nm to 1 nm (e.g., in a membrane or solid media), although smaller and larger diameters may be used. In some embodiments, a micropore comprises a diameters ranging from 100 nm to 100 μm, although smaller and larger diameters may be used.

Nanopores may be biological, e.g., proteinaceous, or solid-state (e.g., including high-density materials such as silicon oxides and nitrides, and polymeric materials such as hard plastics). Exemplary nanopores are described, for example, in U.S. Pat. Nos. 6,746,594, 6,673,615, 6,627,067, 6,464,842, 6,362,002, 6,267,872, 6,015,714, and 5,795,782 and U.S. Publication Nos. 2004/0121525, 2003/0104428, and 2003/0104428, each of which is hereby incorporated by reference in its entirety. An exemplary method for fabricating solid-state membranes is the ion beam sculpting method described in Li et al. Nature 412:166 (2001) and in Chen et al. Nano Letters 4:1333 (2004), each of which is hereby incorporated by reference in its entirety. For the applications of DNA sequencing, several methods have been disclosed that utilize nanopores. U.S. Pat. No. 5,795,782, issued to Church et al. discloses a method of reading DNA sequence by detecting the ionic current variations as a single-stranded DNA molecule moving through the nanopore under a bias voltage. Another method for DNA sequencing using nanopores is described in U.S. Pat. No. 6,537,755, issued to Drmanac. Drmanac uses nanopores to detect the DNA hybridization probes (oligonucleotides) on a DNA molecule and recover the DNA sequence information using the method of Sequencing-By-Hybridization (SBH).

The invention is not limited to using nanopores to detect nucleic acids. For example, other applications using nanopores have been discussed widely in the art, including real-time monitoring of cell activities and detection of biological agents in biodefense. Thus, in some embodiments, the invention provides, in addition to being useful for rapid DNA sequencing, devices and systems for use in a variety of applications involving, but not limited to, single-molecule biophysics, molecular biology, and biochemistry. For example, nanopore devices and systems of the present invention are useful as a molecular comb to probe the secondary structure of RNA molecules, and for use in detecting biological warfare agents, contaminants and pollutants in air and/or water. According to an aspect of the invention, there is featured an electrically-addressable nanopore array that is configured and arranged so as to allow high throughput analyses of biomolecules as well as sequencing DNA.

In some embodiments, an electrically addressable nanopore array includes an insulating material layer. In some embodiments, the insulating material layer is formed from a first sub-layer and a second sub-layer that are bonded or secured to each other using any of a number of techniques known to those skilled in the art and appropriate for the materials being used so as to form the insulating material layer. In an exemplary, illustrative embodiment, the material layer comprises a single silicon wafer. In another illustrative embodiment, the material layer comprises two silicon wafers bonded together.

In some embodiments, the invention provides a device/system comprising a nanopore or plurality of nanopores wherein the device comprises cis and trans chambers connected by an electrical communication means. At the cis end of the electrical communication means is a horizontal aperture sealed with a thin film that includes a single nanopore or channel or plurality of the same. The devices further include a means for applying an electric field between the cis and trans chambers. The subject devices find use in applications in which the ionic current through a nanopore or channel is monitored where such applications include the characterization of naturally occurring ion channels, the characterization of polymeric compounds, and the like.

Inserted into the horizontal bilayer is a single channel or nanopore through which ionic current can flow, e.g. from the cis to the trans side of the pore upon application of an applied electric field. As used herein, the terms “nanopore” and “channel” are used interachangeably to refer to structures having a nanoscale passageway through which ionic current can flow. The inner diameter of the nanopore may vary considerably depending on the intended use of the device. Typically, the channel or nanopore will have an inner diameter of at least about 0.5 nm, usually at least about 1 nm and more usually at least about 1.5 nm, where the diameter may be as great as 50 nm or longer, but in many embodiments will not exceed about 10 nm, and usually will not exceed about 2 nm. In those preferred embodiments in which the subject device is designed to characterize polymeric molecules, the inner diameter of the nanopore may be sufficient to allow translocation of singled stranded, but not double stranded, nucleic acids.

The nanopore should allow a sufficiently large ionic current under an applied electric field to provide for adequate measurement of current fluctuations. As such, under an applied electric field of 120 mV (e.g., in the presence of pH 7.5 buffered solution), an open, unobstructed nanopore should provide for an ionic current that is at least about 1 pA, usually at least about 10 pA and more usually at least about 100 pA. Typically, the ionic current under these conditions will not exceed about 0.5 nA and more usually will not exceed about 1 nA. In addition, the channel should provide for a stable ionic current over a relatively long period of time. Generally, channels finding use in the subject devices provide for accurate measurement of ionic current for at least about 1 minute, usually at least about 10 minutes and more usually at least about 1 hour, where they may provide for a stable current for as long as 24 hours or longer.

Nanopores that are inserted into the lipid bilayer may be a naturally occurring or synthetic nanopore. Typically the nanopore will be a proteinaceous material, by which is meant that it is made up of one or more, usually a plurality, of different proteins associated with each other to produce a channel having an inner diameter of appropriate dimensions, as described above. Suitable channels or nanopores include porins, gramicidins, and synthetic peptides.

Where a device is used to characterize the properties of a naturally occurring ion channel, the nanopore that is inserted into or present in a lipid bilayer covering a substrate and/or aperture is the ion channel of interest. The ionic current through the ion channel is then measured under various conditions, e.g. in the presence of various buffer solutions, agents, lipid bilayers and the like, so as to characterize the ion channel (See, e.g., Wonderlin et al., “Optimizing planar lipid bilayer single-channel recordings for high resolution with rapid voltage steps” Biophys. J. (1990) 58:289-297; and Brutyan et al., “Horizontal ‘solvent-free’ lipid bimolecular membranes with two-sided access can be formed and facilitate ion channel reconstitution,” Biochimica et Biophysica Acta, (1995) 1236: 339-344).

As described above, a device/system of the invention also find use in methods of characterizing polymeric molecules, e.g. determining the sequence of bases in a given nucleic acid. In such methods, the polymer is moved relative to the nanopore in a manner such that each different monomeric unit of the polymer causes a correspondingly different current to flow through the nanopore. For example, a single stranded nucleic acid may be translocated through the nanopore and the effect of each base on the current flowing through the nanopore monitored and recorded. From the resultant recorded current fluctuations, the base sequence of the nucleic acid is determined

While there described herein certain specific embodiments of the invention, it will be manifest to those skilled in the art that various modifications and rearrangements of the parts may be made without departing from the spirit and scope of the underlying inventive concept and that the same is not limited to the particular forms herein described.

1. A device comprising one or more nanopores associated with one or more osmoprotectants.

2. The device of claim 1, wherein the nanopores are in physical contact with one or more osmoprotectants.

3. The device of claim 1, wherein the nanopores are utilized for sensing or characterizing molecules.

4. The device of claim 3, wherein the molecules are nucleic acid.

5. The device of claim 1, wherein the one or more osmoprotectants allows the device to be utilized in a manner not possible in the absence of the osmoprotectant.

6. The device of claim 5, wherein the manner is operation of the device at a high salt concentration.

7. The device of claim 1, wherein the osmoprotectant is selected from the group consisting of glycinebetaine, trehalose, sorbitol, sucrose, and 2-o-a-o-glucopyranosyl glycerol.

8. The device of claim 1, wherein the osmoprotectant is betaine.

9. The device of claim 8, wherein the betaine is trimethylglycine.

10. The device of claim 8, wherein the betaine is a phosphonium betaine.

11. The device of claim 1, wherein the diameter of the nanopore is between 100 nm and 1 nm.

12. The device of claim 1, wherein a time-dependent transport property of the nanopore is superior in the presence of the one or more osmoprotectants compared to the property in the absence of the one or more osmoprotectants.

13. The device of claim 12, wherein the time-dependent transport property is selected from the group consisting of current, conductance, resistance, capacitance, charge, concentration, optical property, and chemical structure.

14. The device of claim 1, wherein the one or more osmoprotectants allows a device comprising a nanopore or plurality of nanopores to operate at a higher efficiency than when in the absence of an osmoprotectant.

15. The device of claim 1, wherein the device comprises a nanopore complex.

16. The device of claim 15, wherein the complex comprises a substrate with a nanopore formed therein.

17. The device of claim 16, further comprising a nucleic acid within the nanopore.

18. The device of claim 15, wherein the complex comprises a lipid bilayer.

19. The device of claim 15, wherein the complex comprises an enzyme.

20. The device of claim 16, wherein the substrate is silicon, mica, polyimide or lipid.

21-23. (canceled)
